(19) United States
(12) Patent Application Publication
(10) Pub. No.: US 2010/0158964 A1
(43) Pub. Date: Jun. 24, 2010

(54) Personal Care Composition Providing Quietness and Softness Enhancement and Articles Using the Same

(76) Inventors: Corey Thomas Cunningham, Larsen, WI (US); Scott W. Wenzel, Neenah, WI (US); Christopher Vincent Decker, Neenah, WI (US); Philip Eugene Kieffer, Winneconne, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/641,659

(22) Filed: Dec. 18, 2009

Related U.S. Application Data
(63) Continuation-in-part of application No. 12/341,406, filed on Dec. 22, 2008.

(51) Int. Cl. A61K 9/70; A61K 8/92

(52) U.S. Cl. 424/402; 514/557; 514/506

(57) ABSTRACT

The present disclosure generally relates to personal care compositions and personal care products. More particularly, the disclosure relates to personal care compositions and personal care products that impart perceivable aesthetic benefits of increased softness, quietness and drapability to the skin or hair of a user. To achieve the perceivable aesthetic benefit, a liquid composition containing water, a skin aesthetic agent selected from fatty acids, fatty alcohols, fatty acid derivatives, fatty alcohol derivatives, and/or combinations thereof, an acidifying agent, and an emulsifying agent may be incorporated into the personal care compositions and personal care products.

### RELATED APPLICATION DATA

This application is a continuation-in-part application claiming priority from presently copending U.S. application Ser. No. 12/341,406 entitled “PERSONAL CARE COMPOSITION PROVIDING QUIETNESS AND SOFTNESS ENHANCEMENT, METHODS OF PREPARING, AND ARTICLES USING THE SAME” filed on Dec. 14, 2008, in the names of Christopher V. Decker et al.

### BACKGROUND

Wipes have been used in the personal care industry for numerous years, and generally have a low surfactant, high water base for cleaning bodily fluids or wiping up menses. In recent years, however, consumers have begun demanding more out of personal care products, including wipes. For example, various wipes have come into the market containing ingredients for softer wipes or containing actives for disinfecting surfaces.

Another example of a desired wipe property is the delivery of perceivable consumer aesthetics such as softness. Prior attempts to overcome the difficulties involved in incorporating hydrophobic skin benefit agents into aqueous wet wipe solutions include, for example, solubilizing, dispersing, or microemulsifying oils into a wet wipe solution. These techniques have proven very difficult, however, since stability of an oil in water system is extremely difficult to achieve due to separation of the oil.

The separation issues may be addressed by raising the surfactant concentration in the wet wipe solution, or by incorporating surfactants high in polyethylene glycol (PEG) and/or polypropylene glycol (PPG) to stabilize the oil in the aqueous wet wipe solution over long periods of time. While these approaches may be effective at stabilizing the oil present in the wet wipe solution, there are other drawbacks. In particular, increasing the concentration of surfactant may result in increased irritation to the skin. Additionally, surfactants containing PEG and/or PPG have recently received negative attention from consumer groups.

The use of humectants such as glycerin to achieve good skin feel by inclusion in a wipe solution has also been achieved. Although humectants mix easily into water, they generally need to be included in the composition in high levels to achieve the desired benefit, and these high levels can lead to tack or drag, which is not aesthetically pleasing to the consumer.

There thus exists a need for an additive for personal care products and compositions that can easily disperse or dissolve in the composition, while providing consumer perceptible aesthetic benefits.

### SUMMARY

The present disclosure generally relates to personal care compositions and personal care products. More particularly, the disclosure relates to personal care compositions and personal care products that impart improved softness, drapability, and quietness characteristics. To achieve the perceivable aesthetic benefits, a liquid composition containing water, a skin aesthetic agent selected from fatty acids, fatty esters, fatty alcohols, fatty acid derivatives, fatty ester derivatives, fatty alcohol derivatives, and/or combinations thereof, an acidifying agent, and an emulsifying agent may be incorporated into the personal care compositions and personal care products.

A wet wipe that imparts a perceivable aesthetic benefit to skin contains a wipe substrate and a liquid composition having at least one skin aesthetic agent selected from fatty acids, fatty esters, fatty alcohols fatty acid derivatives, fatty ester derivatives, fatty alcohol derivatives, and/or combinations thereof, is disclosed. The skin aesthetic agent has been protonated by the addition of at least one acidifying agent to the liquid composition.

To obtain the skin aesthetic agent at least one skin aesthetic agent is provided that is selected from fatty acids, fatty esters, fatty alcohols, fatty acid derivatives, fatty ester derivatives, fatty alcohol derivatives, and/or combinations thereof. The at least one skin aesthetic agent is saponified with an alkaline material to form the deprotonated skin aesthetic agent.

A skin aesthetic agent that is already deprotonated may be provided. For example, a skin aesthetic agent that is a carboxylic acid, alcohol, or salt derivative of a fatty acid, a fatty ester, or a fatty alcohol may be provided. Examples of these skin aesthetic agents include potassium soyate, potassium cocoate, potassium rapeseedate, potassium sunflowerate, potassium olivate, potassium palmate, potassium avocadoate, potassium shea butterate, potassium canoloate, potassium safflowerate, potassium oryzarate, potassium ricinoleate potassium babassuate, potassium behenate, potassium caprylate, potassium castorate, potassium caprate, potassium cocoa butterate, potassium dilinoleate, potassium grapeseedate, potassium hempseedate, potassium hydrogenated cocoate, potassium hydrogenated palmate, potassium hydrogenated tallowate, potassium jojobate, potassium lanolate, potassium lardate, potassium laurate, potassium linoleate, potassium macadamiaseedate, potassium mangoseedate, potassium myristate, potassium oleate, potassium palmitate, potassium palm kernelate, potassium passiflora edulis seedate, potassium stearate, potassium sweet almondate, potassium peanutate, potassium tallate, potassium tallowate, potassium tamanuseedate, potassium tsubakiate, potassium undecylenate, sodium soyate, sodium cocoate, sodium rapeseedate, sodium sunflowerate, sodium olivate, sodium palmate, sodium avocadoate, sodium shea butterate, sodium canoloate, sodium safflowerate, sodium oryzarate, sodium ricinoleate, babassuate, sodium behenate, sodium caprylate, sodium castorate, sodium caprate, sodium cocoa butterate, sodium dilinoleate, sodium grapeseedate, sodium hempseedate, sodium hydrogenated cocoate, sodium hydrogenated palmate, sodium hydrogenated tallowate, sodium jojobate, sodium lanolate, sodium lardate, sodium laurate, sodium linoleate, sodium macadamiaseedate, sodium mangoseedate, sodium myristate, sodium oleate, sodium palmitate, sodium palm kernelate, sodium passiflora edulis seedate, sodium stearate, sodium sweet almondate, sodium peanutate, sodium tallate, sodium tallowate, sodium tamanuseedate, sodium tsubakiate, sodium undecylenate, ammonium soyate, ammonium cocoate, ammonium rapeseedate, ammonium sunflowerate, ammonium olivate, ammonium palmate, ammonium avocadoate, ammonium shea butterate, ammonium canoloate, ammonium safflowerate, ammonium oryzarate, ammonium ricinoleate, ammonium babassuate, ammonium behenate, ammonium caprylate, ammonium castorate, ammonium caprate, ammonium cocoa butterate, ammonium dilinoleate, ammonium grapeseedate, ammonium hempseedate, ammonium hydrogenated cocoate, ammonium hydrogenated palmate, ammonium hydrogenated tallowate, ammonium jojobate, ammonium lanolate, ammonium lardate, ammonium laurate, ammonium linoleate, ammonium macadamiaseedate, ammonium mangoseedate, ammonium myristate, ammonium oleate, ammonium palmitate, ammonium palm kernelate, ammonium passiflora edulis seedate, ammonium stearate, ammonium sweet almondate, ammonium peanutate, ammonium tallate, ammonium tallowate, ammonium tamanuseedate, ammonium tsubakiate, ammonium undecylenate and combinations thereof.

As noted above, the liquid composition may include an acidifying agent to protonate the at least one skin aesthetic agent. The acidifying agent may be selected from hydrochloric acid, nitric acid, sulfuric acid, phosphoric acid, acetic acid, propanoic acid, citric acid, malic acid, maleic acid, sorbic acid, ascorbic acid, dehydroacetic acid, benzoic acid, chlorobenzoic acid, chloroacetic acid, dichloroacetic acid, trichloroacetic acid, trifluoroacetic acid, lactic acid, glycolic acid, tartaric acid, oxalic acid, acetoacetic acid, betaine, crotonic acid, glyceric acid, dimethylmaleic acid, malonic acid, glutaric acid, succinic acid, dimethylsuccinic acid, adipic acid, azelaic acid, and combinations thereof.

The liquid composition further may have a pH of less than 6. Desirably, the pH of the liquid composition is between 3.5 and 5.5.

The at least one skin aesthetic agent may have between 1 to 6 sites of unsaturation per molecule. In addition, the at least one skin aesthetic agent may have between 8 and 22 carbon atoms per molecule.

The at least one skin aesthetic agent may be derived from a natural plant source and may be selected from fats, oils, essential oils, essential fatty acids, non-essential fatty acids, and combinations thereof.

The emulsifying agent is selected from carbon based emulsifiers, silicon based emulsifiers, non-ionic emulsifiers, cationic emulsifiers, and combinations thereof. In some embodiments, the emulsifying agent has an HLB value between 11 and 20 and acts as an oil-in-water emulsifier. In other embodiments, the emulsifying agent has an HLB value between 1 and 5 and acts as a secondary emulsifying agent.

The liquid composition may also contain salt. If included, the liquid composition contains salt in amount between about 1% and 4%.

### DETAILED DESCRIPTION

The present disclosure generally relates to personal care compositions and personal care products. More particularly, the disclosure relates to personal care compositions and personal care products that impart a perceivable aesthetic benefit to the skin of a user. To achieve the perceivable aesthetic benefit, a protonated skin aesthetic agent selected from fatty acids, fatty esters, fatty alcohols, fatty acids derivatives, fatty ester derivatives, fatty alcohol derivatives, and/or combinations thereof, may be incorporated into the personal care compositions and personal care products.

It is often desirable for personal care products to deliver good aesthetics to the skin that are perceivable by the consumer. However, prior attempts to improve the aesthetics of substrates, such as wet wipes, have proven difficult. In particular, many skin benefit agents that may act to improve the feel of the wipes are hydrophobic, and thus are difficult to effectively incorporate into wet wipe formulations which typically contain large amounts of water. Other skin benefit agents, such as humectants, will readily mix with water but need to be incorporated into the wet wipe formulation at high levels in order to be effective, which may result in a tacky or sticky feeling wipe.

In accordance with the present disclosure, it has now been discovered that a perceivable aesthetic benefit may be imparted to the skin using a personal care product which includes a liquid composition having a protonated skin aesthetic agent selected from fatty acids, fatty alcohols, fatty acid derivatives, fatty alcohol derivatives, and/or combinations thereof. Advantageously, the skin aesthetic agents used herein have been saponified and thus can readily be incorporated into water-based compositions, such as wet wipe compositions. After adding the deprotonated skin aesthetic agent to the liquid composition, an emulsifying agent is added. An acid is then added to the liquid composition and the acidity of the liquid composition increases and an oil-phase of a protonated skin aesthetic agent is emulsified within the liquid composition.

Furthermore, the presence of the protonated skin aesthetic agent in the liquid composition imparts improved softness, drapability, and quietness characteristics to the product. Unlike other liquid compositions which may contain large amounts of humectant or oil and may feel tacky or greasy on the skin, the compositions of the present disclosure have good tactile properties without leaving a greasy feeling on the skin.

A method of preparing the skin aesthetic agent for use in the liquid composition is also disclosed. To prepare, a skin aesthetic agent selected from fatty acids, fatty esters, fatty alcohols, fatty acid derivatives, fatty ester derivatives, fatty alcohol derivatives, and/or combinations thereof is selected for use in the liquid composition. Many of these skin aesthetic agents are oil based, and generally not soluble in water.

Thus, to prepare a wet wipe that imparts a perceivable aesthetic benefit to skin, a deprotonated skin benefit is provided. A skin aesthetic agent may first be saponified into a deprotonated skin aesthetic agent. The expression saponify or saponification means the hydrolysis of the esters present in, for example, natural fats and oils, and fatty esters, or the neutralization or deprotonation of carboxylic acids and alcohols present in, for example, fatty acids and fatty alcohols, with typical alkaline materials to form an alcohol and a deprotonated carboxylic acid or deprotonated alcohol. As an example, the deprotonated skin aesthetic agent prepared by saponification of the skin benefit agent may be a carboxylate salt. The alkaline materials may include aqueous solutions of an alkali or base such as, for example, sodium hydroxide, potassium hydroxide, ammonium hydroxide, triethylamine, diethylamine, triethanolamine, diethanolamine, calcium hydroxide, lithium hydroxide, barium hydroxide, soda ash, and the like. Alternatively, a skin aesthetic agent may be provided that is already deprotonated. For example, a carboxylic acid derivative of a fatty acid, a fatty ester, or a fatty alcohol such as a carboxylate salt may be provided.

The liquid composition also contains an emulsifying agent. As mentioned above, the fatty acids, esters and alcohols and their derivatives, and combinations thereof, may act as emulsifiers in the composition. The composition may contain an additional emulsifying agent other than the fatty acids, esters and alcohols and their derivatives, and combinations thereof. Any suitable emulsifier may be included in the liquid compositions including carbon based emulsifiers, silicon based emulsifiers, non-ionic emulsifiers, cationic emulsifiers, and combinations thereof. The composition of the present disclosure may suitably include one or more additional emulsifier in an amount of from about 0.01% by weight of the composition to about 10% by weight of the composition.

For example, nonionic surfactants may be used as an emulsifying agent. Nonionic surfactants typically have a hydrophobic base, such as a long chain alkyl group or an alkylated aryl group, and a hydrophilic chain comprising a certain number (e.g., 1 to about 30) of ethoxy and/or propoxy moieties. Examples of some classes of nonionic surfactants that can be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈₋₁₀ acids, condensation products of ethylene oxide with long chain amines or amides, condensation products of ethylene oxide with alcohols, and mixtures thereof.

Various specific examples of suitable nonionic surfactants for use as the emulsifying agent include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, ethoxylated fatty (C₁₋₂₂) alcohol, including 3 to 20 ethylene oxide moieties, polyoxyethylene-20 isohexadecyl ether, polyoxyethylene-23 glycerol laurate, PEG-80 sorbitan laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG-12dioleate, PEG-8dioleate, and combinations thereof.

Desirably, the emulsifying agents will have a hydrophilic/lipophilic balance (HLB) of from 11 to 20, and behave as oil-in-water emulsifying agents. In other embodiments, the emulsifying agents will have a hydrophilic/lipophilic balance (HLB) of from 1 to 5, and behave as a secondary emulsifying agent to the skin aesthetic agent. Addition of an emulsifying agent acts to stabilize the skin aesthetic agent in the liquid composition for better delivery on the wet wipe article during use.

Suitable carbon based emulsifers include polyglyceryl-4 isostearate. Silicon based water-in-oil emulsifers include alkylene oxide graft modified silicone oils. Suitable alkylene oxides include ethylene oxide, propylene oxide, butylene oxide, and mixtures thereof. Grafting of alkylene oxide groups may occur randomly or in blocks. Preferably, the grafting is block grafting of ethylene oxide and propylene oxide in a weight ratio of 10 to 1.

Specific emulsifying agents include Bis-PEG/PPG-14/14 dimethicone (ABIL®EM 97, available from Evonik), polyglyceryl-3 oleate (ISOLAN®GO 33, available from Evonik), polyglyceryl-4 diisostearate/polyhydroxystearate/sebacate (ISOLAN®GPS, available from Evonik), cetearyl alcohol, dicetyl phosphate, and ceteth-10 phosphate (Crodafos CES, available from Croda) polyglyceryl-2 dipolyhydroxystearate, polyglyceryl-3 polyricinoleate, PEG-30 dipolyhydroxystearate, glyceryl stearate, hydrogenated vegetable glycerides phosphate, palmitamidopropyltrimonium chloride, sorbitan oleate, sorbitan sesquioleate, sorbitan isostearate, sorbitan trioleate, polyglyceryl-3-diisostearate, polyglyceryl-4 oleate, cetearyl alcohol/dicetyl phosphate/ceteth-10 phosphate blends, PPG-5 ceteth-20, palmitamidopropyltrimonium chloride, polyglyceryl-5 laurate, glyceryl stearate/PEG-100 stearate blends, sorbitan trioleate, hydroxypropyl bis-hydroxyethyldimonium chloride, cetearyl alcohol/PEG-40 castor oil/sodium cetearyl sulfate blends, and combinations thereof.

The liquid compositions may also further contain preservatives. Suitable preservatives for use in the present compositions may include, for instance, Kathon CG®, which is a mixture of methylchloroisothiazolinone and methylisothiazolinone available from Rohm & Haas; Neolone 950®, which is methylisothiazolinone available from Rohm & Haas, DMDM hydantoin (e.g., Glydant Plus available from Lonza, Inc. of Fair Lawn, N.J.); iodopropynyl butylcarbamate; benzoic esters (parabens), such as methylparaben, propylparaben, butylparaben, ethylparaben, isopropyl paraben, isobutylparaben, benzylparaben, sodium methylparaben, and sodium propylparaben; 2-bromo-2-nitropropane-1,3-diol; benzoic acid; imidazolidinyl urea; diazolidinyl urea; and the like. Still other preservatives may include ethylhexylglycerin (Sensiva SC 50 by Schulke & Mayr), phenoxyethanol (Phenoxyethanol by Tri-K Industries), caprylyl glycol (Lexgard O by Inolex Chemical Company, Symdiol 68T (a blend of 1,2-hexanediol, caprylyl glycol and tropolone by Symrise) and Symocide PT (a blend of phenoxyethanol and tropolone by Symrise).

The deprotonated skin aesthetic agent is then added to an aqueous liquid composition. The deprotonated skin aesthetic agent is water soluble and is easily added to the aqueous liquid composition. An acidifying agent is then added to the aqueous liquid composition acidifying the aqueous liquid composition and protonating the skin aesthetic agent.

To prepare a wet wipe, the aqueous liquid solution containing the emulsion of the protonated skin aesthetic agent is incorporated onto a wipe substrate.

As an example, soybean oil may be used as the skin aesthetic agent to impart softness, quietness and drapability to a wipe. The soya fatty acids present in soybean oil are not soluble in water. Therefore, the soya oil is saponified with potassium hydroxide to form potassium soyate, the potassium salt of soya fatty acid. The water-soluble potassium soyate is then added to an aqueous liquid composition. An emulsifying agent and an acidifying agent are then added to the liquid composition to produce an emulsion and protonate the potassium soyate within the liquid composition.

Desirably, the skin aesthetic agent has between 8 to 30 carbon atoms per molecule. It has been discovered that a skin aesthetic agent with a smaller carbon chain provides better softness, quietness and drapability in a wiping application. Thus even more desirably, the skin aesthetic agent has between 8 to 22 carbon atoms per molecule.

Desirably, the skin aesthetic agents have between 1 to 6 sites of unsaturation. It has been discovered that a skin aesthetic agent with a higher amount of unsaturation provides better softness, quietness and drapability in a wiping application.

To protonate the skin aesthetic agent, at least one acidifying agent is used with the liquid composition. The acidifying agent must be capable of reducing the pH of the liquid composition below the pKa of the skin aesthetic agent. Exemplary acidifying agents include inorganic acids and organic acids. Suitable acids include, but are not limited to, hydrochloric acid, nitric acid, sulfuric acid, phosphoric acid, acidacetic acid, propanoic acid, citric acid, malic acid, maleic acid, sorbic acid, ascorbic acid, dehydroacetic acid, benzoic acid, chlorobenzoic acid, chloroacetic acid, dichloroacetic acid, trichloroacetic acid, trifluoroacetic acid, lactic acid, glycolic acid, tartaric acid, oxalic acid, acetoacetic acid, betaine, crotonic acid, glyceric acid, dimethylmaleic acid, malonic acid, glutaric acid, succinic acid, dimethylsuccinic acid, adipic acid, azelaic acid, and combinations thereof.

In an exemplary aspect, after acidifying, the liquid composition has a pH of less than about 6. Desirably, the liquid composition has a pH of between about 3.5 and 5.5.

The at least one protonated skin aesthetic agent may be a fatty ester or fatty ester derivative that has been saponified and then protonated into a fatty acid or fatty alcohol. The fatty esters would first need to be saponified as described above to make them water soluble. The saponification converts the esters to deprotonated fatty acids in an aqueous solution. When the pH of the aqueous solution is lowered, the deprotonated fatty acids are then converted to protonated fatty acids, creating an oil phase that is emulsified within the liquid composition. In particular, the at least one protonated skin aesthetic agent is a triglyceride ester derivative that has been saponified and then protonated into a fatty acid or fatty alcohol.

Exemplary fatty esters for use as a skin aesthetic agent may include, but are not limited to, octyldodecyl neopentanoate, stearyl stearate, isopropyl myristate, isopropyl palmitate, stearyl behenate, C₁₂₋₁₅ alkyl benzoate, butyl isostearate, cetyl caprate, cetyl caprylate, ethyl apricot kernelate, ethyl avocadate, ethylhexyl caprate/caprylate, ethylhexyl cocoate, ethylhexyl isopalmitate, isocetyl myristate, isopropyl jojobate, myristyl laurate, and combinations thereof. Exemplary fatty acids for use as a skin aesthetic agent may include, but are not limited to, palmitic acid, stearic acid, myristic acid, oleic acid, linoleic acid, linolenic acid, behenic acid, arachadonic acid, behenoyl stearic acid and combinations thereof. Exemplary fatty alcohols for use as a skin aesthetic agent could include, but are not limited to, octyldodecanol, lauryl, myristyl, cetyl, stearyl, behenyl alcohol, and combinations thereof. Additionally, combinations of different fatty alcohols, fatty esters and fatty acids may be used.

The protonated skin aesthetic agents may be derived from 100% natural fats and oils. As used herein, the term “natural fat or oil” is intended to include fats, oils, essential oils, essential fatty acids, non-essential fatty acids, and combinations thereof, that are derived from natural plant sources. The oils would first need to be saponified as described above to make them water soluble. The saponification converts the glycerides in the oils to deprotonated fatty acids in an aqueous solution. When the pH of the aqueous solution is lowered, the deprotonated fatty acids are then converted to protonated fatty acids, creating an oil phase emulsified within the liquid composition.

Suitable natural fats or oils can include citrus oil, olive oil, avocado oil, apricot oil, babassu oil, borage oil, camellia oil, canola oil, castor oil, coconut oil, corn oil, cottonseed oil, evening primrose oil, hydrogenated cottonseed oil, hydrogenated palm kernel oil, jojoba oil, maleated soybean oil, meadowfoam seed oil, palm kernel oil, peanut oil, rapeseed oil, grapeseed oil, safflower oil, sweet almond oil, tall oil, lauric acid, myristic acid, palmitic acid, stearic acid, oleic acid, linoleic acid, linolenic acid, stearyl alcohol, lauryl alcohol, myristyl alcohol, behenyl alcohol, rose hip oil, calendula oil, chamomile oil, eucalyptus oil, juniper oil, sandalwood oil, tea tree oil, sunflower oil, soybean oil, poppy seed oil, hazelnut oil, walnut oil, tall oil, sesame seed oil, rice bran oil, wheat germ oil, linseed oil, tung oil, ongokea oil, hemp oil, tsubakiate oil, shea butter, cocoa butter, mango seed butter, and combinations thereof.

Fatty acid, alcohol and salt derivatives of various fatty acids, fatty esters and fatty alcohols, and combinations thereof, may be used for the liquid composition. Desirably, carboxylates of fatty acids, fatty esters and fatty alcohols, and combinations thereof, are used as the skin aesthetic agent with the liquid composition. The carboxylates have been protonated by neutralizing and acidifying the liquid composition with an acidifying agent.

Natural fats and oils are treated with aqueous solutions of hydroxide salts. Suitable hydroxide salts include, but are not limited to, sodium, potassium, calcium, ammonium, tetrabutylammonium, and combinations thereof. In an exemplary aspect, the skin aesthetic agent may be selected from potassium soyate, potassium cocoate, potassium rapeseedate, potassium sunflowerate, potassium olivate, potassium palmate, potassium avocadoate, potassium shea butterate, potassium canoloate, potassium safflowerate, potassium oryzarate, potassium ricinoleate, potassium babassuate, potassium behenate, potassium caprylate, potassium castorate, potassium caprate, potassium cocoa butterate, potassium dilinoleate, potassium grapeseedate, potassium hempseedate, potassium hydrogenated cocoate, potassium hydrogenated palmate, potassium hydrogenated tallowate, potassium jojobate, potassium lanolate, potassium lardate, potassium laurate, potassium linoleate, potassium macadamiaseedate, potassium mangoseedate, potassium myristate, potassium oleate, potassium palmitate, potassium palm kernelate, potassium passiflora edulis seedate, potassium stearate, potassium sweet almondate, potassium peanutate, potassium tallate, potassium tallowate, potassium tamanuseedate, potassium tsubakiate, potassium undecylenate, sodium soyate, sodium cocoate, sodium rapeseedate, sodium sunflowerate, sodium olivate, sodium palmate, sodium avocadoate, sodium shea butterate, sodium canoloate, sodium safflowerate, sodium oryzarate, sodium ricinoleate, sodium babassuate, sodium behenate, sodium caprylate, sodium castorate, sodium caprate, sodium cocoa butterate, sodium dilinoleate, sodium grapeseedate, sodium hempseedate, sodium hydrogenated cocoate, sodium hydrogenated palmate, sodium hydrogenated tallowate, sodium jojobate, sodium lanolate, sodium lardate, sodium laurate, sodium linoleate, sodium macadamiaseedate, sodium mangoseedate, sodium myristate, sodium oleate, sodium palmitate, sodium palm kernelate, sodium passiflora edulis seedate, sodium stearate, sodium sweet almondate, sodium peanutate, sodium tallate, sodium tallowate, sodium tamanuseedate, sodium tsubakiate, sodium undecylenate, ammonium soyate, ammonium cocoate, ammonium rapeseedate, ammonium sunflowerate, ammonium olivate, ammonium palmate, ammonium avocadoate, ammonium shea butterate, ammonium canoloate, ammonium safflowerate, ammonium oryzarate, ammonium ricinoleate, ammonium babassuate, ammonium behenate, ammonium caprylate, ammonium castorate, ammonium caprate, ammonium cocoa butterate, ammonium dilinoleate, ammonium grapeseedate, ammonium hempseedate, ammonium hydrogenated cocoate, ammonium hydrogenated palmate, ammonium hydrogenated tallowate, ammonium jojobate, ammonium lanolate, ammonium lardate, ammonium laurate, ammonium linoleate, ammonium macadamiaseedate, ammonium mangoseedate, ammonium myristate, ammonium oleate, ammonium palmitate, ammonium palm kernelate, ammonium passiflora edulis seedate, ammonium stearate, ammonium sweet almondate, ammonium peanutate, ammonium tallate, ammonium tallowate, ammonium tamanuseedate, ammonium tsubakiate, ammonium undecylenate and combinations thereof. Desirably, potassium soyate or potassium cocoate may be protonated with an acidifying agent to form an oil phase that is emulsified within the liquid composition.

The liquid composition may include skin aesthetic agents in an amount of from about 0.01% by weight of the composition to about 20% by weight of the composition, more desirably from about 0.05% by weight of the composition to about 15% by weight of the composition, and even more desirably from about 0.1% by weight of the composition to about 10% by weight of the composition.

The skin aesthetic agents may be used in combination with a product, such as a personal care product. More particularly, the skin aesthetic agents may be incorporated into a liquid composition that may be incorporated into or onto a substrate, such as a wipe substrate, a fabric or cloth substrate, or a tissue substrate, among others. For example, the liquid compositions may be incorporated into personal care products, such as wipes, bath tissues, cloths, and the like. More particularly, use of a skin aesthetic agent-containing composition may be incorporated into wipes such as wet wipes, dry wipes, hand wipes, face wipes, cosmetic wipes, and the like. Desirably, the skin aesthetic agents are contained within a liquid composition that may be used in combination with a wipe substrate to form a wet wipe, or may be a wetting composition for use in combination with a dispersible wet wipe.

As noted above, the skin aesthetic agents may be incorporated into liquid compositions and wipes to improve the perceivable aesthetics of these products. The present disclosure may be directed to wipes. Generally, the wipes of the present disclosure including the protonated fatty acids, fatty esters and fatty alcohols can be wet wipes or dry wipes. As used herein, the term “wet wipe” means a wipe that includes greater than about 70% by weight of the substrate moisture content. As used herein, the terms “dry wipe” and “substantially dry wipe”, used interchangeably herein, mean a wipe that includes less than about 10% by weight of the substrate moisture content. Specifically, suitable wipes for use in the present disclosure can include wet wipes, dry wipes, hand wipes, face wipes, cosmetic wipes, household wipes, industrial wipes, and the like. Particularly preferred wipes are wet wipes, and other wipe-types that include a solution.

Materials suitable for the substrate of the wipes are well known to those skilled in the art, and are typically made from a fibrous sheet material which may be either woven or nonwoven. For example, suitable materials for use in the wipes may include nonwoven fibrous sheet materials which include meltblown, coform, air-laid, bonded-carded web materials, hydroentangled materials, and combinations thereof. Such materials can be made of synthetic or natural fibers, or a combination thereof. Typically, the wipes of the present disclosure define a basis weight of from about 25 grams per square meter to about 120 grams per square meter and desirably from about 40 grams per square meter to about 90 grams per square meter.

In one particular aspect, the wipes of the present disclosure are constructed of a coform basesheet of polymer fibers and absorbent fibers having a basis weight of from about 45 to about 80 grams per square meter and desirably about 60 grams per square meter. Typically, such coform basesheets are constructed of a gas-formed matrix of thermoplastic polymeric meltblown fibers and cellulosic fibers. Various suitable materials may be used to provide the polymeric meltblown fibers, such as, for example, polypropylene microfibers. Alternatively, the polymeric meltblown fibers may be elastomeric polymer fibers, such as those provided by a polymer resin. For instance, Vistamaxx® elastic olefin copolymer resin designated PLTD-1810, available from ExxonMobil Corporation of Houston, Tex., or KRATON G-2755, available from Kraton Polymers of Houston, Tex., may be used to provide stretchable polymeric meltblown fibers for the coform basesheets. Other suitable polymeric materials or combinations thereof may alternatively be utilized as known in the art.

The coform basesheet additionally may be constructed of various absorbent cellulosic fibers, such as, for example, wood pulp fibers. Suitable commercially available cellulosic fibers for use in the coform basesheets can include, for example, NF 405, which is a chemically treated bleached southern softwood Kraft pulp, available from Weyerhaeuser Co. of Washington, D.C.; NB 416, which is a bleached southern softwood Kraft pulp, available from Weyerhaeuser Co.; CR-0056, which is a fully debonded softwood pulp, available from Bowater, Inc. of Greenville, SC; Golden Isles 4822 debonded softwood pulp, available from Koch Cellulose of Brunswick, Ga.; and SULPHATATE HJ, which is a chemically modified hardwood pulp, available from Rayonier, Inc. of Jesup, Ga.

The relative percentages of the polymeric meltblown fibers and cellulosic fibers in the coform basesheet can vary over a wide range depending upon the desired characteristics of the wipes. For example, the coform basesheet may have from about 10 weight percent to about 90 weight percent, desirably from about 20 weight percent to about 60 weight percent, and more desirably from about 25 weight percent to about 35 weight percent of polymeric meltblown fibers based on the dry weight of the coform basesheet being used to provide the wipes.

The wipe substrate may also be an airlaid nonwoven fabric. The basis weights for airlaid nonwoven fabrics may range from about 20 to about 200 grams per square meter (gsm) with staple fibers having a denier of about 0.5 to 10 and a length of about 6 to 15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

The wipes can be formed from a composite which includes multiple layers of materials. For example, the wipes may include a three layer composite which includes an elastomeric film or meltblown layer between two coform layers as described above. In such a configuration, the coform layers may define a basis weight of from about 15 grams per square meter to about 30 grams per square meter and the elastomeric layer may include a film material such as a polyethylene metallocene film.

As mentioned above, one type of wipe suitable for use in combination with the liquid composition is a wet wipe. In addition to the wipe substrate, wet wipes also contain a liquid wipe composition described herein. The liquid wet wipe composition can be any liquid, which can be absorbed into the wet wipe basesheet and may include any suitable components, which provide the desired wiping properties. For example, the components may include water, emollients, surfactants, fragrances, preservatives, organic or inorganic acids, chelating agents, pH buffers, or combinations thereof, as are well known to those skilled in the art. Further, the liquid may also contain lotions, medicaments, and/or antimicrobials.

The liquid composition may desirably be incorporated into the wipe in an add-on amount of from about 10% by weight of the substrate to about 600% by weight of the substrate, more desirably from about 50% by weight of the substrate to about 500% by weight of the substrate, even more desirably from about 100% by weight of the substrate to about 500% by weight of the substrate, and especially more desirably from about 200% by weight of the substrate to 300% by weight of the substrate.

The desired liquid wet wipe composition add-on amounts may vary depending on the composition of the wipe substrate. Typically, however, for coform basesheets, the wet-wipe composition add-on amount will be from about 250% by weight of the substrate to about 350% by weight of the substrate, and more typically about 330% by weight of the substrate. For air-laid basesheets, the composition add-on amount will typically be from about 130% by weight of the substrate to about 300% by weight of the substrate, and more typically will be about 235% by weight of the substrate.

These add-on amounts will preferably result in a wet wipe having the skin aesthetic agents in an add-on amount of from about 0.1% by weight of the substrate to about 10% by weight of the substrate, and more preferably from about 0.3% by weight of the substrate to about 4.95% by weight of the substrate. The add-on amounts of the skin aesthetic agents will depend on the concentration of the skin aesthetic agents in the wet wipe composition and the total add-on amount of the composition.

The personal care product may also be a dry substrate. The personal care product can be wetted with an aqueous solution just prior to, or at the point of, use of the dry substrate. The aqueous solution can be any aqueous solution known in the art to be suitable for use in wipe products. Generally, the aqueous solution includes mainly water, and can further include additional components, such as cleansers, lotions, preservatives, fragrances, surfactants, emulsifiers, dyes, humectants, emollients, oils, sunscreens, and combinations thereof. The natural fatty acids, esters and alcohols and their derivatives, and combinations thereof, may be present in the aqueous solution used to wet the dry wipe prior to use.

Alternately, the dry substrate may be prepared by applying, by any suitable means (e.g., by spraying, impregnating, etc.), a liquid composition having protonated fatty acids, fatty esters, fatty alcohols, derivatives thereof, and/or combinations thereof, onto a dry substrate. The composition may contain 100% of the skin aesthetic agents or alternately, skin aesthetic agents may be present in the composition in combination with a carrier and/or other skin benefit agent, as described herein. In the embodiments where the liquid composition used to prepare the dry wipe contains water or moisture, the resulting treated substrate is then dried so that the substrate contains less than about 10% moisture content by weight of the substrate, and a dry substrate is produced. The treated substrate can be dried by any means known to those skilled in the art including, for example, by use of convection ovens, radiant heat sources, microwave ovens, forced air ovens, heated rollers or cans, or combinations thereof.

The dry substrate may contain the liquid composition in an add-on amount of composition of from about 40% by weight of the treated substrate to about 250% by weight of the treated substrate, more typically from about 75% by weight of the treated substrate to about 150% by weight of the treated substrate and more typically about 100% by weight of the treated substrate.

As noted above, the skin aesthetic agents may be incorporated into personal care products such as wipes or tissues to improve the perceivable aesthetics of the product. One example of a perceivable aesthetic benefit achieved by incorporating skin aesthetic agents into a wipe is improved softness of the wipe across the skin as compared to traditional wipe products.

In particular, cup crush values can be used as an indication of softness of materials that may contact the skin, such as a wipe. Lower cup crush values indicate an increased feeling of gentleness of the wipe and softness of the wipe as it glides across the skin.

Typically, the cup crush value for a wipe incorporating skin aesthetic agents of the present disclosure will be from about 800 to about 1100 gf*mm. Dynamic cup crush values may be measured as described in the examples.

Coefficient of friction (MUI) and surface softness (MMD) values can be used as an indication of softness of materials that may contact the skin, such as a wipe. Lower coefficient of friction values (MIU) indicate less drag and friction on the sample surface. Lower values of surface softness (MMD) indicate less variation or more uniformity on the sample surface. Both values indicate an increased feeling of gentleness of the wipe and softness of the wipe as it glides across the skin.

Typically, the coefficient of friction (MUI) value for a wipe incorporating skin aesthetic agents of the present disclosure will be less than about 0.30. Coefficient of friction (MUI) values may be measured as described in the examples.

Typically, the surface softness (MMD) value for a wipe incorporating skin aesthetic agents of the present disclosure will be less than about 0.0075. Surface softness (MMD) values may be measured as described in the examples.

In addition to increased gentleness and improved glide of a wipe across the skin, incorporating skin aesthetic agents into a liquid composition or wipe will also reduce the level of noise that may otherwise occur when the wipe is being used. Typically, the sound-level value for a wipe incorporating skin aesthetic agents of the present disclosure will be from about 10.5 to about 15.2 dB.

An increase in sound-level of about 3 dB results in doubling of the sound intensity. Therefore, the noise difference is perceptible by a consumer during normal wiping. A wipe having a lower noise-level will allow the wipe to seem softer and gentler to a consumer. Sound-level values may be measured as described in the examples.

Inclusion of skin aesthetic agents into a wipe, such as a wet wipe, may desirably allow the wipe to better drape over the surface of the hand. Drapability is a measurement of the relative stiffness or softness of a substrate. Increased drapability provides for easier use of the wipe. Lower bending stiffness and lower bending hysteresis values indicate higher drapability. Lower values of bending stiffness indicate lower stiffness of the samples and hence higher flexibility. Lower values of bending hysteresis indicate higher ability for the samples to recover from the bending action.

Typically, the bending stiffness for a wipe incorporating skin aesthetic agents will be from about 0.03 to 0.059 gf*cm²/cm. Typically, the bending hysteresis for a wipe incorporating skin aesthetic agents will be from about 0.07 to 0.09 gf*cm/cm.

Non-limiting examples of suitable carrier materials include water; glycols such as propylene glycol, butylene glycol, and ethoxydiglycol; lower chain alcohols such as ethanol and isopropanol; glycerin and glycerin derivatives; natural oils such as jojoba oil and sunflower oil; synthetic oils such as mineral oil; silicone derivatives such as cyclomethicone, and other pharmaceutically acceptable carrier materials. As will be recognized by one skilled in the art, the relative amounts of carrier material and other components in the compositions that can be used to formulate the composition will be dictated by the nature of the composition. The levels can be determined by routine experimentation in view of the disclosure provided herein.

The liquid compositions desirably contain water. The liquid compositions can suitably contain water in an amount of from about 0.1% by weight of the composition to about 99.9% by weight of the composition, more typically from about 40% by weight of the composition to about 99.9% by weight of the composition, and more preferably from about 60% by weight of the composition to about 99.9% by weight of the composition. For instance, where the composition is used in connection with a wet wipe, the composition can suitably contain water in an amount of from about 75% by weight of the composition to about 99.9% by weight of the composition.

The liquid composition may also contain salt. Desirably, if salt is included in the composition, the liquid composition contains salt in amount between about 1% and 4%.

The liquid compositions may further contain additional agents that impart a beneficial effect on skin or hair and/or further act to improve the aesthetic feel of the compositions and wipes described herein. Examples of suitable skin benefit agents include emollients, sterols or sterol derivatives, natural and synthetic fats or oils, viscosity enhancers, rheology modifiers, polyols, surfactants, alcohols, esters, silicones, clays, starch, cellulose, particulates, moisturizers, film formers, slip modifiers, surface modifiers, skin protectants, humectants, sunscreens, and the like.

Thus, in one aspect, the liquid compositions may further optionally include one or more emollient, which typically acts to soften, soothe, and otherwise lubricate and/or moisturize the skin. Suitable emollients that can be incorporated into the compositions include oils such as petrolatum based oils, petrolatum, mineral oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, glycerol esters and derivatives, propylene glycol esters and derivatives, alkoxylated carboxylic acids, alkoxylated alcohols, and combinations thereof.

Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof, can also suitably be used as emollients.

In instances wherein the liquid composition is used in combination with a wet wipe, the composition may include an emollient in an amount of from about 0.01% by weight of the composition to about 20% by weight of the composition, more desirably from about 0.05% by weight of the composition to about 10% by weight of the composition, and more typically from about 0.1% by weight of the composition to about 5% by weight of the composition.

One or more viscosity enhancers may also be added to the liquid composition to increase the viscosity, to help stabilize the composition, such as when the composition is incorporated into a personal care product, thereby reducing migration of the composition and improving transfer to the skin. Suitable viscosity enhancers include polyolefin resins, lipophilic/oil thickeners, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, clays, starches, gums, water-soluble acrylates, carbomers, acrylate based thickeners, surfactant thickeners, and combinations thereof.

The liquid composition may desirably include one or more viscosity enhancers in an amount of from about 0.01% by weight of the composition to about 25% by weight of the composition, more desirably from about 0.05% by weight of the composition to about 10% by weight of the composition, and even more desirably from about 0.1% by weight of the composition to about 5% by weight of the composition.

The liquid compositions may optionally further contain humectants. Examples of suitable humectants include glycerin, glycerin derivatives, sodium hyaluronate, betaine, amino acids, glycosaminoglycans, honey, sorbitol, glycols, polyols, sugars, hydrogenated starch hydrolysates, salts of PCA, lactic acid, lactates, and urea. A particularly preferred humectant is glycerin. The liquid composition may suitably include one or more humectants in an amount of from about 0.05% by weight of the composition to about 25% by weight of the composition.

The liquid compositions may optionally further contain film formers. Examples of suitable film formers include lanolin derivatives (e.g., acetylated lanolins), superfatted oils, cyclomethicone, cyclopentasiloxane, dimethicone, synthetic and biological polymers, proteins, quaternary ammonium materials, starches, gums, cellulosics, polysaccharides, albumen, acrylates derivatives, IPDI derivatives, and the like. The liquid composition may suitably include one or more film formers in an amount of from about 0.01% by weight of the composition to about 20% by weight of the composition.

The liquid compositions may optionally further contain slip modifiers. Examples of suitable slip modifiers include bismuth oxychloride, iron oxide, mica, surface treated mica, ZnO, ZrO₂, silica, silica silyate, colloidal silica, attapulgite, sepiolite, starches (i.e., corn, tapioca, rice), cellulosics, nylon-12, nylon-6, polyethylene, talc, styrene, polystyrene, polypropylene, ethylene/acrylic acid copolymer, acrylates, acrylate copolymers (i.e., methylmethacrylate crosspolymer), sericite, titanium dioxide, aluminum oxide, silicone resin, barium sulfate, calcium carbonate, cellulose acetate, polymethyl methacrylate, polymethylsilsequioxane, talc, tetrafluoroethylene, silk powder, boron nitride, lauroyl lysine, synthetic oils, natural oils, esters, silicones, glycols, and the like. The liquid composition may suitably include one or more slip modifier in an amount of from about 0.01% by weight of the composition to about 20% by weight of the composition.

The liquid compositions may also further contain surface modifiers. Examples of suitable surface modifiers include silicones, quaternium materials, powders, salts, peptides, polymers, clays, and glyceryl esters. The liquid composition may suitably include one or more surface modifier in an amount of from about 0.01% by weight of the composition to about 20% by weight of the composition.

The liquid compositions may also further contain skin protectants. Examples of suitable skin protectants include ingredients referenced in SP monograph (21 CFR §347). Suitable skin protectants and amounts include those set forth in SP monograph, Subpart B—Active Ingredients §347.10: (a) Allantoin, 0.5 to 2%, (b) Aluminum hydroxide gel, 0.15 to 5%, (c) Calamine, 1 to 25%, (d) Cocoa butter, 50 to 100%, (e) Cod liver oil, 5 to 13.56%, in accordance with §347.20(a)(1) or (a)(2), provided the product is labeled so that the quantity used in a 24-hour period does not exceed 10,000 U.S.P. Units vitamin A and 400 U.S.P. Units cholecalciferol, (f) Colloidal oatmeal, 0.007% minimum; 0.003% minimum in combination with mineral oil in accordance with §347.20(a)(4), (g) Dimethicone, 1 to 30%, (h) Glycerin, 20 to 45%, (i) Hard fat, 50 to 100%, (j) Kaolin, 4 to 20%, (k) Lanolin, 12.5 to 50%, (l) Mineral oil, 50 to 100%; 30 to 35% in combination with colloidal oatmeal in accordance with §347.20(a)(4), (m) Petrolatum, 30 to 100%, (O) Sodium bicarbonate, (q) Topical starch, 10 to 98%, (r) White petrolatum, 30 to 100%, (s) Zinc acetate, 0.1 to 2%, (t) Zinc carbonate, 0.2 to 2%, (u) Zinc oxide, 1 to 25%.

The liquid compositions may also further contain quaternary ammonium materials. Examples of suitable quaternary ammonium materials include polyquaternium-7, polyquaternium-10, benzalkonium chloride, behentrimonium methosulfate, cetrimonium chloride, cocamidopropyl pg-dimonium chloride, guar hydroxypropyltrimonium chloride, isostearamidopropyl morpholine lactate, polyquaternium-33, polyquaternium-60, polyquaternium-79, quaternium-18 hectorite, quaternium-79 hydrolyzed silk, quaternium-79 hydrolyzed soy protein, rapeseed amidopropyl ethyldimonium ethosulfate, silicone quaternium-7, stearalkonium chloride, butylglucosides, hydroxypropyltrimonium chloride, laurdimoniumhydroxypropyl decylglucosides chloride, and the like. The composition of the present disclosure may suitably include one or more quaternary materials in an amount of from about 0.01% by weight of the composition to about 20% by weight of the composition.

The liquid compositions may optionally further contain surfactants. Examples of suitable additional surfactants include, for example, anionic surfactants, cationic surfactants, amphoteric surfactants, zwitterionic surfactants, non-ionic surfactants, and combinations thereof. Specific examples of suitable surfactants are known in the art and include those suitable for incorporation into liquid compositions and wipes. The liquid composition may suitably include one or more surfactants in an amount of from about 0.01% by weight of the composition to about 20% by weight of the composition.

In addition to nonionic surfactants, the cleanser may also contain other types of surfactants. For instance, in some embodiments, amphoteric surfactants, such as zwitterionic surfactants, may also be used. For instance, one class of amphoteric surfactants that may be used in the present disclosure are derivatives of secondary and tertiary amines having aliphatic radicals that are straight chain or branched, wherein one of the aliphatic substituents contains from about 8 to 18 carbon atoms and at least one of the aliphatic substituents contains an anionic water-solubilizing group, such as a carboxy, sulfonate, or sulfate group. Some examples of amphoteric surfactants include, but are not limited to, sodium 3-(dodecylamino)propionate, sodium 3-(dodecylamino)-propane-1-sulfonate, sodium 2-(dodecylamino)ethyl sulfate, sodium 2-(dimethylamino)octadecanoate, disodium 3-(N-carboxymethyl-dodecylamino)propane-1-sulfonate, disodium octadecyliminodiacetate, sodium 1-carboxymethyl-2-undecylimidazole, and sodium N,N-bis(2-hydroxyethyl)-2-sulfato-3-dodecoxypropylamine.

Additional classes of suitable amphoteric surfactants include phosphobetaines and the phosphitaines. For instance, some examples of such amphoteric surfactants include, but are not limited to, sodium coconut N-methyl taurate, sodium oleyl N-methyl taurate, sodium tall oil acid N-methyl taurate, sodium palm itoyl N-methyl taurate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl)carboxymethylbetaine, oleyldimethylgammacarboxypropylbetaine, lauryl-bis-(2-hydroxypropyl)-carboxyethylbetaine, cocoamidodimethylpropylsultaine, stearylamidodimethylpropylsultaine, laurylamido-bis-(2-hydroxyethyl)propylsultaine, di-sodium oleamide PEG-2 sulfosuccinate, TEA oleamido PEG-2 sulfosuccinate, disodium oleamide MEA sulfosuccinate, disodium oleamide MIPA sulfosuccinate, disodium ricinoleamide MEA sulfosuccinate, disodium undecylenamide MEA sulfosuccinate, disodium lauryl sulfosuccinate, disodium wheat germamido MEA sulfosuccinate, disodium wheat germamido PEG-2 sulfosuccinate, disodium isostearamideo MEA sulfosuccinate, cocoamphoglycinate, cocoamphocarboxyglycinate, lauroamphoglycinate, lauroamphocarboxyglycinate, capryloamphocarboxyglycinate, cocoamphopropionate, cocoamphocarboxypropionate, lauroamphocarboxypropionate, capryloamphocarboxypropionate, dihydroxyethyl tallow glycinate, cocoamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, cocamidopropyl betaine, lauric myristic amido propyl monosodium phosphitaine, and mixtures thereof.

In certain instances, it may also be desired to utilize one or more anionic surfactants within the cleansers. Suitable anionic surfactants include, but are not limited to, alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylauryl sulfonates, alkyl monoglyceride sulfates, alkyl monoglyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxyethylene sulfates, isethionates, or mixtures thereof.

Particular examples of some suitable anionic surfactants include, but are not limited to, C₈₋₁₈ alkyl sulfates, C₈₋₁₈ fatty acid salts, C₈₋₁₈ alkyl ether sulfates having one or two moles of ethoxylation, C₈₋₁₈ alkamine oxides, C₈₋₁₈ alkoyl sarcosinates, C₈₋₁₈ sulfoacetates, C₈₋₁₈ sulfosuccinates, C₈₋₁₈ alkyl diphenyl oxide disulfonates, C₈₋₁₈ alkyl carbonates, C₈₋₁₈ alpha-olefin sulfonates, methyl ester sulfonates, and blends thereof. The C₈₋₁₈ alkyl group can be straight chain (e.g., lauryl) or branched (e.g., 2-ethylhexyl). The cation of the anionic surfactant can be an alkali metal (e.g., sodium or potassium), ammonium, C₁₋₄ alkylammonium (e.g., mono-, di-, tri-), or C₁₋₃ alkanolammonium (e.g., mono-, di-, tri-).

Specific examples of such anionic surfactants include, but are not limited to, lauryl sulfates, octyl sulfates, 2-ethylhexyl sulfates, lauramine oxide, decyl sulfates, tridecyl sulfates, cocoates, lauroyl sarcosinates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, stearates, tallates, ricinoleates, cetyl sulfates, and similar surfactants.

Cationic surfactants, such as cetylpyridinium chloride and methylbenzethonium chloride, may also be utilized.

The liquid compositions may additionally include adjunct components conventionally found in pharmaceutical compositions in their art-established fashion and at their art-established levels. For example, the compositions may contain additional compatible pharmaceutically active materials for combination therapy, such as antimicrobials, antioxidants, anti-parasitic agents, antipruritics, antifungals, antiseptic actives, biological actives, astringents, keratolytic actives, local anesthetics, anti-stinging agents, anti-reddening agents, skin soothing agents, and combinations thereof. Other suitable additives that may be included in the liquid compositions include colorants, deodorants, fragrances, perfumes, emulsifiers, anti-foaming agents, lubricants, natural moisturizing agents, skin conditioning agents, skin protectants and other skin benefit agents (e.g., extracts such as aloe vera and anti-aging agents such as peptides), solvents, solubilizing agents, suspending agents, wetting agents, humectants, pH adjusters, buffering agents, dyes and/or pigments, and combinations thereof.

### EXAMPLES

The following non-limiting examples are provided to further illustrate the present disclosure.

Test Methods

Cud Crush: As used herein, the term “cup crush” refers to one measure of the softness of a nonwoven fabric sheet that is determined according to the “cup crush” test. The test is generally performed as discussed in detail in U.S. patent application Ser. No. 09/751,329 entitled, “Composite Material With Cloth-Like Feel” filed Dec. 29, 2000, hereby incorporated by reference. The cup crush test evaluates fabric stiffness by measuring the peak load (also called the “cup crush load” or just “cup crush”) required for a 4.5 cm diameter hemispherically shaped foot to crush a 17.8 cm by 17.8 cm piece of fabric shaped into an approximately 6.5 cm diameter by 6.5 cm tall cup shape, while the now cup shaped fabric is surrounded by an approximately 6.5 cm diameter cylinder cup to maintain a uniform deformation of the cup shaped fabric. There can be gaps between a ring and the forming cup, but at least four corners of the fabric must be fixedly pinched there between. The foot and cylinder cup are aligned to avoid contact between the cup walls and the foot that could affect the readings. The load is measured in grams, and recorded a minimum of twenty times per second while the foot is descending at a rate of about 406 mm per minute. The cup crush test provides a value for the total energy required to crush a sample (the “cup crush energy”) which is the energy over a 4.5 cm range beginning about 0.5 cm below the top of the fabric cup, i.e., the area under the curve formed by the load in grams on one axis and the distance the foot travels in millimeters on the other. Cup crush energy is reported in gm-mm (or inch-pounds). A lower cup crush value indicates a softer material. A suitable device for measuring cup crush is a model FTD-G-500 load cell (500 gram range) available from the Schaevitz Company of Pennsauken, N.J.

Sound-Level: As used herein, “sound-level” refers to a measurement of the amount of noise transmitted by a substrate passing over on a surface in a suitable sound chamber. A suitable sound chamber is discussed in detail in U.S. patent application Ser. No. 10/719,639 entitled, “Reduced-Noise Composite Materials and Disposable Personal Care Devices Employing the Same” filed Nov. 21, 2003, hereby incorporated by reference. The testing apparatus includes a sound chamber and a sound level meter. The purpose of the apparatus is to manipulate an article in a controlled noise environment, and to accurately quantify the noise produced by the movement of the article.

The sound chamber includes a door, a top wall, a bottom wall, two side walls, and a rear wall. The door and each wall are constructed of 0.25 inch (0.635 cm) thick 6061 grade anodized aluminum. The door and rear wall are each 36 inches (91.4 cm) in height and 24 inches (61.0 cm) in width. The test chamber side walls are each 36 inches (91.4 cm) high and 18 inches (45.7 cm) wide. The test chamber top and bottom panels are each 24 inches wide (61.0 cm) and 18 inches (45.7 cm) long. The interior surface of the door and each wall has applied thereto two-inch thick polyurethane sound-dampening foam (available from Illbruck Inc. of Minneapolis, Minn., under the brand name SONEX and stock number SOC-2). As shown, a sound level meter support extends perpendicularly outward from the side wall just below a microphone orifice.

The microphone orifice is positioned 14.5 centimeters above the floor of the bottom wall, and is further centered between the door and the rear wall. The sound level meter support is constructed of aluminum and is bolted to the side wall.

A sound level meter, such as a model 1900, equipped with a model OB-100 octave filter set (available from Quest Technologies of Oconomowoc, Wis.). The sound level meter is supported by a model QC-20 calibrator and QuestSuite master module software, each also available from Quest Technologies. During operation of the testing apparatus, the sound level meter rests in the sound level meter support. The sound level meter includes a microphone extending 4.75 inches (12 centimeters) therefrom. The octave filter is set to 2 kHz.

First, measurements of the background noise are measured with the sound level meter to find a background sound-level. Then, a forearm is held in the sound chamber, perpendicular to the front face of the sound chamber. The center of the forearm is approximately 12 cm away from the interior side of the wall of the sound chamber. This measurement is made from the wall of the sound chamber containing the microphone orifice for the sound level meter. A substrate sample is tested by wiping on the volar forearm, at a rate of approximately 1 second per forearm length. Approximately, 15 seconds of data was taken for each sample. The sound level reader takes a measurement every second. Three samples are tested for each example. The sound level value is calculated from the average decibel level of the 3 samples and subtracting the background sound level.

Bending Test: Bending stiffness and hysteresis was measured using the KES model FB-2 (available from the Kato Tech Co, Ltd. of Japan). To measure bending the sample is clamped in an upright position between two chucks and a 0.4 mm center adjustment plate is used (the size of the adjustment plate is dependent on the sample thickness). One of the chucks is stationary while the other rotates in a curvature between 2.5 cm⁻¹ and −2.5 cm⁻¹. The bending tester (KES-FB-2) measures the pure bending properties of a sample for a given range of curvature at a constant rate of 0.5 cm⁻¹/s. The bending curvature 0 to 2.5 cm⁻¹ denotes as the forward bend while bending through curvature 0 to −2.5 cm⁻¹ denotes as the backward bend. The movable chuck moves at a rate of 0.5 cm⁻¹/sec. The amount of moment (grams force*cm/cm) taken to bend the material vs. the curvature is plotted. For all the materials tested, the following instrument settings were used:

Measurement mode=one cycle

Sensitivity=2×1

K Span Control=SET

Curvature=+/−2.5 cm⁻¹

The KES system algorithm computes the following bending characteristic values:

B=bending stiffness (grams force×cm²/cm)

2HB=bending hysteresis (grams force×cm/cm)

The bending stiffness is defined as the slope of bending moment versus curvature taken between 0.5 cm⁻¹ and 1.5 cm⁻¹, and bending hysteresis is a measure of recovery of the sample after it has been bent and is the distance of the bending and recovery curves at the curvature of 1.0 cm⁻¹. The samples are tested along MD and CD for 5 times each. Higher values bending stiffness indicate higher stiffness of the samples. Higher values of bending hysteresis indicate more difficult for the samples to recover from the bending action.

Surface Test: Coefficient of friction (MUI) and surface softness (MMD) refer to measures of the softness of a nonwoven fabric sheet that is determined according to the “surface test.” Machine Direction Coefficient of Friction and Cross-Machine Direction of Coefficient of Friction is obtained using the Kawabata Evaluation System (KES) test instrument model KES-SE (available from Kato Tech Co, Ltd. of Japan). The sample is placed on a specimen tray, and a holding frame is placed over the specimen. The machine direction measurement is taken first. Two probes are placed on the sample. The coefficient of friction is measured using probes with 10 pieces of steel wires each 0.5 mm in diameter, and is designed to simulate the human finger. The sample is moved forward and backward underneath the two probes at a constant rate of 0.1 cm/sec. The measurement is taken for 2 cm over the surface. The distance or displacement of the probes is detected by a potentiometer. The coefficient of friction probes is detected by a force transducer. The displacement (distance) of the sample (L, cm) vs. the coefficient of friction (MIU-unitless) is plotted. A value for surface softness (MMD-unitless) is the mean deviation of MIU. The sample is then rotated 90 degrees and tested again to provide the cross-machine direction measurements. The following settings were used:

Friction sensitivity=2×5

Roughness Sensitivity=2×5

Static Load=25 g

With the above settings, the raw numbers from the instrument are then multiplied by 0.2 to yield the final coefficient of friction results.

Lower values of coefficient of friction (MIU) indicate less drag on the sample surface. Lower values of surface softness (MMD) indicate less variation or more uniformity on the sample surface T.

$$MIU ( \overline{μ} ) = \frac{1}{X} {\int }_{0}^{x} μ d x$$

$$MMD = \frac{1}{X} {\int }_{0}^{x} \vert μ - \overline{μ} \vert d x$$

where

μ=friction force divided by compression force

μ̅=mean value of μ

x=displacement of the probe on the surface of specimen, cm

X=maximum travel used in the calculation, 2 cm

T=thickness of specimen at position x, micron

T̅=mean value of T, micron

### Example 1

In this example, liquid compositions were prepared using a skin aesthetic agent. The composition components are listed in Table 1 and Table 2.

TABLE 1
Exemplary Composition A

| Trade Name | INCI Name | Wt. % | Grams |
|---|---|---|---|
| Water | Water | 98.45 | 492.25 |
| Potassium Soyate | Potassium Soyate | 1.000 | 5.00 |
| Sodium Benzoate | Sodium Benzoate | 0.450 | 2.25 |
| Neolone 950 | Methylisothiazolinone | 0.100 | 0.50 |
| Malic Acid | Malic Acid | q.s. pH 5.5 | q.s. pH 5.5 |

Exemplary Composition A was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform, followed by addition of any remaining composition components. The pH of Exemplary Composition A was adjusted to about pH 5.5 using malic acid, as needed. Exemplary Composition A was then coated on 60 gsm (grams per square meter) coform baby wipes at 330% add-on.

TABLE 2
Exemplary Composition B

| Trade Name | INCI Name | Wt. % | Grams |
|---|---|---|---|
| Water | Water | 97.45 | 487.3 |
| Potassium | Potassium Cocoate | 2.000 | 10.00 |
| Cocoate | | | |
| Sodium Benzoate | Sodium Benzoate | 0.450 | 2.25 |
| Neolone 950 | Methylisothiazolinone | 0.100 | 0.50 |
| Malic Acid | Malic Acid | q.s. pH 5.5 | q.s. pH 5.5 |

Exemplary Composition B was prepared by combining water with preservatives sodium benzoate and methylisothiazolinone followed by mixing until uniform. The skin aesthetic agent, potassium cocoate (commercially available from Lubrizol Corp. of Houston, Tex.), was then added, and the resulting mixture was mixed until uniform, followed by addition of any remaining composition components. The pH of Exemplary Composition B was adjusted to about 5.5 using malic acid, as needed. Exemplary Composition B was then coated on 60 gsm (grams per square meter) coform baby wipes at 330% add-on.

### Example 2

In this example, the sound-level of wet wipes having a skin aesthetic agent of the present disclosure was determined using the method described in the Test Methods section. Wet wipes were prepared as described in Example 1. Additionally, the wetting compositions used to prepare the wet wipes had added thereto a skin aesthetic agent of the present disclosure (Exemplary Compositions A and B). A control code was tested that did not have a skin aesthetic agent of the present disclosure, but did contain a surfactant blend (Comparative Composition C). A surfactant blend containing potassium laureth phosphate, glycerin, polysorbate 20, tetrasodium EDTA, methylparaben, methylisothiazolinone, aloe barbadensis leaf extract, and tocopheryl acetate was prepared (wipes solution commercially available from Kimberly-Clark Corporation). Approximately 4.15% of the surfactant blend by weight was added into water and then coated on 60 gsm (grams per square meter) coform baby wipes at 330% add-on.

The type and amount of additive and results are set forth in Table 3.

TABLE 3
Sound-Level Results

| | Skin Aesthetic | Sound-Level |
|---|---|---|
| Composition | Agent (% by weight) | (dB) |
| A | Potassium Soyate (1%) | 15.0 |
| B | Potassium Cocoate (2%) | 11.4 |
| C | Surfactant Blend | 19.6 |

As can be seen from these results, the presence of a skin aesthetic agent in a wet wipe composition lowers the sound intensity of the wipe passing on a substrate.

### Example 3

In this example, the cup-crush value of wet wipes having a skin aesthetic agent of the present disclosure was determined using the method described in the Test Methods section. Wet wipes were prepared as described in Example 1. Additionally, the wetting compositions used to prepare the wet wipes had added thereto a skin aesthetic agent of the present disclosure (Exemplary Compositions A and B). A control code was tested that did not have a skin aesthetic agent of the present disclosure, but did contain a surfactant blend (Comparative Composition C). A surfactant blend containing potassium laureth phosphate, glycerin, polysorbate 20, tetrasodium EDTA, methylparaben, methylisothiazolinone, aloe barbadensis leaf extract, and tocopheryl acetate was prepared (wipes solution commercially available from Kimberly-Clark Corporation). Approximately 4.15% of the surfactant blend by weight was added into water and then coated on 60 gsm (grams per square meter) coform baby wipes at 330% add-on.

The type and amount of additive and results are set forth in Table 4.

TABLE 4
Cup-Crush Test Results

| | Skin Aesthetic | Cup Crush |
|---|---|---|
| Composition | Agent (% by weight) | (gf * mm) |
| A | Potassium Soyate (1%) | 1106 |
| B | Potassium Cocoate (2%) | 1044 |
| C | Surfactant Blend | 1398 |

As can be seen from these results, the presence of about 0-2% of a skin aesthetic agent in a wet wipe composition lowers the cup crush of the wipe substrate. It is believed that increasing the amount of skin aesthetic agent in Exemplary Composition A would also lower the cup crush of this sample.

### Example 4

In this example, the coefficient of friction (MUI) and surface softness (MMD) of wet wipes having a skin aesthetic agent of the present disclosure was determined using the surface test method described in the Test Methods section. Wet wipes were prepared as described in Example 1. Additionally, the wetting compositions used to prepare the wet wipes had added thereto a skin aesthetic agent of the present disclosure (Exemplary Compositions A and B). A control code was tested that did not have a skin aesthetic agent of the present disclosure, but did contain a surfactant blend (Comparative Composition C). A surfactant blend containing potassium laureth phosphate, glycerin, polysorbate 20, tetrasodium EDTA, methylparaben, methylisothiazolinone, aloe barbadensis leaf extract, and tocopheryl acetate was prepared (wipes solution commercially available from Kimberly-Clark Corporation). Approximately 4.15% of the surfactant blend by weight was added into water and then coated on 60 gsm (grams per square meter) coform baby wipes at 330% add-on.

The type and amount of additive and results are set forth in Table 5.

TABLE 5
Surface Test Results

| Composition | Skin Aesthetic Agent (% by weight) | MUI | MMD |
|---|---|---|---|
| A | Potassium Soyate (1%) | 0.2320 | 0.00750 |
| B | Potassium Cocoate (2%) | 0.2210 | 0.00756 |
| C | Surfactant Blend | 0.2788 | 0.00910 |

As can be seen from these results, the presence of a skin aesthetic agent in a wet wipe composition lowers the bending stiffness and bending hysteresis of the wipe substrate.

### Example 5

In this example, the bending stiffness and bending hysteresis of wet wipes having a skin aesthetic agent of the present disclosure was determined using the method described in the Test Methods section. Wet wipes were prepared as described in Example 1. Additionally, the wetting compositions used to prepare the wet wipes had added thereto a skin aesthetic agent of the present disclosure (Exemplary Compositions A and B). A control code was tested that did not have a skin aesthetic agent of the present disclosure, but did contain a surfactant blend (Comparative Composition C). A surfactant blend containing potassium laureth phosphate, glycerin, polysorbate 20, tetrasodium EDTA, methylparaben, methylisothiazolinone, aloe barbadensis leaf extract, and tocopheryl Acetate (wipes solution commercially available from Kimberly-Clark Corporation) in water was then coated on 60 gsm (grams per square meter) coform baby wipes at 330% add-on.

The type and amount of additive and results are set forth in Table 6.

TABLE 6
Bending Test Results

| | | Bending | Bending |
|---|---|---|---|
| | Skin Aesthetic | Stiffness | Hysteresis |
| Composition | Agent (% by weight) | (gf * cm²/cm) | (gf * cm/cm) |
| A | Potassium Soyate (1%) | 0.0450 | 0.0765 |
| B | Potassium Cocoate (2%) | 0.0470 | 0.0817 |
| C | Surfactant Blend | 0.0598 | 0.0933 |

As can be seen from these results, the presence of a skin aesthetic agent in a wet wipe composition lowers the bending stiffness and bending hysteresis of the wipe substrate.

### Example 6

In this example, liquid compositions were prepared using a skin aesthetic agent and an emulsifying agent. The composition components are listed in Tables 7-11.

TABLE 7
Exemplary Composition D

| Trade Name | INCI Name | Wt. % |
|---|---|---|
| Water | Water | 98.25 |
| Potassium Soyate | Potassium Soyate | 1.000 |
| Sodium Benzoate | Sodium Benzoate | 0.450 |
| Neolone 950 | Methylisothiazolinone | 0.100 |
| Dermofeel G5L | Polyglyceryl-5 Laurate | 0.125 |
| Malic Acid | Malic Acid | q.s. pH 5.5 |

Exemplary Composition D was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, polyglyceryl-5 laurate, was added and mixed until uniform. The pH of Exemplary Composition D was adjusted to about pH 5.5 using malic acid, as needed. Exemplary Composition D was then coated on 60 gsm (grams per square meter) coform baby wipes at 330% add-on.

TABLE 8
Exemplary Composition E

| Trade Name | INCI Name | Wt. % |
|---|---|---|
| Water | Water | 98.20 |
| Potassium Soyate | Potassium Soyate | 1.00 |
| Sodium Benzoate | Sodium Benzoate | 0.45 |
| Neolone 950 | Methylisothiazolinone | 0.10 |
| Varisoft PATC | Palmitamidopropyltrimonium | 0.25 |
| | Chloride | |
| Malic Acid | Malic Acid | q.s. pH 5.5 |

Exemplary Composition E was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, palmitamidopropyltrimonium chloride, was added and mixed until uniform. The pH of Exemplary Composition E was adjusted to about pH 5.5 using malic acid, as needed. Exemplary Composition E was then coated on 60 gsm (grams per square meter) coform baby wipes at 330% add-on.

TABLE 9
Exemplary Composition F

| Trade Name | INCI Name | Wt. % |
|---|---|---|
| Water | Water | 98.20 |
| Potassium Soyate | Potassium Soyate | 1.00 |
| Sodium Benzoate | Sodium Benzoate | 0.45 |
| Neolone 950 | Methylisothiazolinone | 0.10 |
| Procetyl AWS | PPG-5 Ceteth-20 | 0.25 |
| Malic Acid | Malic Acid | q.s. pH 5.5 |

Exemplary Composition F was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, PPG-5 ceteth-20, was added and mixed until uniform. The pH of Exemplary Composition F was adjusted to about pH 5.5 using malic acid, as needed. Exemplary Composition F was then coated on 60 gsm (grams per square meter) coform baby wipes at 330% add-on.

TABLE 10
Exemplary Composition G

| Trade Name | INCI Name | Wt. % |
|---|---|---|
| Water | Water | 97.95 |
| Potassium Soyate | Potassium Soyate | 1.00 |
| Sodium Benzoate | Sodium Benzoate | 0.45 |
| Neolone 950 | Methylisothiazolinone | 0.10 |
| Arlacel 165 | Glyceryl Stearate/PEG-100 | 1.00 |
| | Stearate Blend | |
| Malic Acid | Malic Acid | q.s. pH 5.5 |

Exemplary Composition G was prepared by combining approximately two-thirds of the total water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Meanwhile, the remaining one-third of the total water was mixed with the emulsifier and heated to 60 C. with stirring until completely dissolved. Finally, the emulsifying agent, the glyceryl stearate and PEG-100 stearate blend, was added and mixed until uniform. The pH of Exemplary Composition G was adjusted to about pH 5.5 using malic acid, as needed. Exemplary Composition G was then coated on 60 gsm (grams per square meter) coform baby wipes at 330% add-on.

TABLE 11
Exemplary Composition H

| Trade Name | INCI Name | Wt. % |
|---|---|---|
| Water | Water | 97.95 |
| Potassium Soyate | Potassium Soyate | 1.00 |
| Sodium Benzoate | Sodium Benzoate | 0.45 |
| Neolone 950 | Methylisothiazolinone | 0.10 |
| Emulgade F | Cetearyl Alcohol/PEG-40 Castor | 1.00 |
| | Oil/Sodium Cetearyl Sulfate | |
| Malic Acid | Malic Acid | q.s. pH 5.5 |

Exemplary Composition H was prepared by combining approximately two-thirds of the total water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Meanwhile, the remaining one-third of the total water was mixed with the emulsifier and heated to 60 C. with stirring until completely dissolved. Finally, the emulsifying agent, a blend of cetearyl alcohol/PEG-40 castor oil/sodium cetearyl sulfate, was added and mixed until uniform. The pH of Exemplary Composition H was adjusted to about pH 5.5 using malic acid, as needed. Exemplary Composition H was then coated on 60 gsm (grams per square meter) coform baby wipes at 330% add-on.

Example Compositions D-H were evaluated in sensory tests and compared to HUGGIES® wet wipes (commercially available from Kimberly-Clark Corporation). The wipes were evaluated for various wiping characteristics, including handheld softness, wiping softness, and cushiony feel. Results from these studies are illustrated in Table 3 and summarized below. For the characteristics, examples with the same letter are similar in that attribute, and examples with letter A are considered to have the most of that characteristic. Treatment levels are the total treatment applied to the 2 sides of the treated tissue.

TABLE 11
Sensory Study Results

| | Handheld | Wiping | |
|---|---|---|---|
| Composition | Softness | Softness | Cushiony |
| D | C | BC | A |
| E | C | BC | AB |
| F | B | AB | AB |
| G | C | C | B |
| H | A | A | AB |
| HUGGIES | E | E | C |

The HUGGIES® wet wipes serve as the performance benchmark for the sensory effects in terms of handheld softness, wiping softness, and cushiony feel. Compositions D-H all have better handheld softness, wiping softness, and cushiony feel. Composition H having potassium soyate as the skin aesthetic agent and the blend of cetearyl alcohol/PEG-40 castor oil/sodium cetearyl sulfate as the emulsifying agent is perceived as having the highest amount of handheld softness, wiping softness, and cushiony feel.

### Example 7

In this example, liquid compositions were prepared using a skin aesthetic agent, an emulsifying agent, and salt. The composition components are listed in Tables 12-15. These compositions are also stable and provide a wipe with a perception of improved aesthetics such as softness.

TABLE 12
Exemplary Composition I

| Trade Name | INCI Name | Wt. % |
|---|---|---|
| Water | Water | 96.33 |
| Potassium Soyate | Potassium Soyate | 1.00 |
| Sodium Benzoate | Sodium Benzoate | 0.45 |
| Neolone 950 | Methylisothiazolinone | 0.10 |
| Sodium Chloride | Sodium Chloride | 2.00 |
| Span 85 | Sorbitan Trioleate | 0.125 |
| Malic Acid | Malic Acid | q.s. pH 5.5 |

Exemplary Composition I was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, sorbitan trioleate, and salt were added and mixed until uniform. The pH of Exemplary Composition I was adjusted to about pH 5.5 using malic acid, as needed. Exemplary Composition I was then coated on 60 gsm (grams per square meter) coform baby wipes at 330% add-on.

TABLE 13
Exemplary Composition J

| Trade Name | INCI Name | Wt. % |
|---|---|---|
| Water | Water | 96.33 |
| Potassium Soyate | Potassium Soyate | 1.00 |
| Sodium Benzoate | Sodium Benzoate | 0.45 |
| Neolone 950 | Methylisothiazolinone | 0.10 |
| Sodium Chloride | Sodium Chloride | 2.00 |
| Arlacel 83 | Sorbitan Sesquioleate | 0.125 |
| Malic Acid | Malic Acid | q.s. pH 5.5 |

Exemplary Composition J was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, sorbitan sesquioleate, and salt were added and mixed until uniform. The pH of Exemplary Composition J was adjusted to about pH 5.5 using malic acid, as needed. Exemplary Composition J was then coated on 60 gsm (grams per square meter) coform baby wipes at 330% add-on.

TABLE 14
Exemplary Composition K

| Trade Name | INCI Name | Wt. % |
|---|---|---|
| Water | Water | 95.95 |
| Potassium Soyate | Potassium Soyate | 1.00 |
| Sodium Benzoate | Sodium Benzoate | 0.45 |
| Neolone 950 | Methylisothiazolinone | 0.10 |
| Sodium Chloride | Sodium Chloride | 2.00 |
| Crodafos CES | Cetearyl Alcohol/Dicetyl | 0.50 |
| | Phosphate/Ceteth-10 Phosphate | |
| Malic Acid | Malic Acid | q.s. pH 5.5 |

Exemplary Composition K was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, cetearyl alcohol/dicetyl phosphate/ceteth-10 phosphate blend, and salt were added and mixed until uniform. The pH of Exemplary Composition K was adjusted to about pH 5.5 using malic acid, as needed. Exemplary Composition K was then coated on 60 gsm (grams per square meter) coform baby wipes at 330% add-on.

TABLE 15
Exemplary Composition L

| Trade Name | INCI Name | Wt. % |
|---|---|---|
| Water | Water | 95.45 |
| Potassium Soyate | Potassium Soyate | 1.00 |
| Sodium Benzoate | Sodium Benzoate | 0.45 |
| Neolone 950 | Methylisothiazolinone | 0.10 |
| Sodium Chloride | Sodium Chloride | 2.00 |
| ColaMoist 200 | Hydroxypropyl Bis- | 1.00 |
| | Hydroxyethyldimonium Chloride | |
| Malic Acid | Malic Acid | q.s. pH 5.5 |

Exemplary Composition L was prepared by combining water with preservatives sodium benzoate and methylisothiazolinone followed by mixing until uniform. The skin aesthetic agent, potassium soyate (commercially available from Lubrizol Corp. of Houston, Tex.), was then added, and the resulting mixture was mixed until uniform. Finally, the emulsifying agent, hydroxypropyl bis-hydroxyethyldimonium chloride, and salt were added and mixed until uniform. The pH of Exemplary Composition L was adjusted to about pH 5.5 using malic acid, as needed. Exemplary Composition L was then coated on 60 gsm (grams per square meter) coform baby wipes at 330% add-on.

Other modifications and variations to the present invention may be practiced by those of ordinary skill in the art, without departing from the spirit and scope of the present invention, which is more particularly set forth in the appended claims. It is understood that aspects of the various embodiments may be interchanged in whole or part. The preceding description, given by way of example in order to enable one of ordinary skill in the art to practice the claimed invention, is not to be construed as limiting the scope of the invention, which is defined by the claims and all equivalents thereto.

1. A wet wipe that imparts a perceivable aesthetic benefit to skin, the wipe comprising:
a wipe substrate; and
a liquid composition containing:
from about 40% by weight of the composition to about 99.9% by weight of the composition of water;
from about 0.01% by weight of the composition to about 20% by weight of the composition of at least one skin aesthetic agent selected from fatty acids, fatty esters, fatty alcohols, fatty acid derivatives, fatty ester derivatives, fatty alcohol derivatives, and/or combinations thereof;
an acidifying agent; and
from about 0.01% by weight of the composition to about 10% by weight of the composition of an emulsifying agent.

2. The wet wipe of claim 1 wherein the at least one skin aesthetic agent has between 1 to 6 sites of unsaturation per molecule and between 8 and 30 carbon atoms per molecule.

3. The wet wipe of claim 1 wherein the acidifying agent protonates the at least one skin aesthetic agent.

4. The wet wipe of claim 3 wherein the liquid composition has a pH between 3.5 and 6.

5. The wet wipe of claim 1 wherein the at least one skin aesthetic agent is a fatty ester or fatty ester derivative that has been saponified and then protonated into a fatty acid or fatty alcohol.

6. The wet wipe of claim 1 wherein the at least one skin aesthetic agent is a triglyceride ester derivative that has been saponified and then protonated into a fatty acid or fatty alcohol.

7. The wet wipe of claim 1 wherein the at least one protonated skin aesthetic agent is a salt derivative of a fatty acid, a fatty ester, or a fatty alcohol selected from potassium soyate, potassium cocoate, potassium rapeseedate, potassium sunflowerate, potassium olivate, potassium palmate, potassium avocadoate, potassium shea butterate, potassium canoloate, potassium safflowerate, potassium oryzarate, potassium ricinoleate, potassium babassuate, potassium behenate, potassium caprylate, potassium castorate, potassium caprate, potassium cocoa butterate, potassium dilinoleate, potassium grapeseedate, potassium hempseedate, potassium hydrogenated cocoate, potassium hydrogenated palmate, potassium hydrogenated tallowate, potassium jojobate, potassium lanolate, potassium lardate, potassium laurate, potassium linoleate, potassium macadamiaseedate, potassium mangoseedate, potassium myristate, potassium oleate, potassium palmitate, potassium palm kernelate, potassium passiflora edulis seedate, potassium stearate, potassium sweet almondate, potassium peanutate, potassium tallate, potassium tallowate, potassium tamanuseedate, potassium tsubakiate, potassium undecylenate, sodium soyate, sodium cocoate, sodium rapeseedate, sodium sunflowerate, sodium olivate, sodium palmate, sodium avocadoate, sodium shea butterate, sodium canoloate, sodium safflowerate, sodium oryzarate, sodium ricinoleate, sodium babassuate, sodium behenate, sodium caprylate, sodium castorate, sodium caprate, sodium cocoa butterate, sodium dilinoleate, sodium grapeseedate, sodium hempseedate, sodium hydrogenated cocoate, sodium hydrogenated palmate, sodium hydrogenated tallowate, sodium jojobate, sodium lanolate, sodium lardate, sodium laurate, sodium linoleate, sodium macadamiaseedate, sodium mangoseedate, sodium myristate, sodium oleate, sodium palmitate, sodium palm kernelate, sodium passiflora edulis seedate, sodium stearate, sodium sweet almondate, sodium peanutate, sodium tallate, sodium tallowate, sodium tamanuseedate, sodium tsubakiate, sodium undecylenate, ammonium soyate, ammonium cocoate, ammonium rapeseedate, ammonium sunflowerate, ammonium olivate, ammonium palmate, ammonium avocadoate, ammonium shea butterate, ammonium canoloate, ammonium safflowerate, ammonium oryzarate, ammonium ricinoleate, ammonium babassuate, ammonium behenate, ammonium caprylate, ammonium castorate, ammonium caprate, ammonium cocoa butterate, ammonium dilinoleate, ammonium grapeseedate, ammonium hempseedate, ammonium hydrogenated cocoate, ammonium hydrogenated palmate, ammonium hydrogenated tallowate, ammonium jojobate, ammonium lanolate, ammonium lardate, ammonium laurate, ammonium linoleate, ammonium macadamiaseedate, ammonium mangoseedate, ammonium myristate, ammonium oleate, ammonium palmitate, ammonium palm kernelate, ammonium passiflora edulis seedate, ammonium stearate, ammonium sweet almondate, ammonium peanutate, ammonium tallate, ammonium tallowate, ammonium tamanuseedate, ammonium tsubakiate, ammonium undecylenate and combinations thereof.

8. The wet wipe of claim 1 wherein the at least one skin aesthetic agent is derived from a natural plant source and selected from fats, oils, essential oils, essential fatty acids, non-essential fatty acids, and combinations thereof.

9. The wet wipe of claim 1 wherein the liquid composition comprises from about 0.1% by weight of the composition to about 10% by weight of the composition of at least one skin aesthetic agent.

10. The wet wipe of claim 1 wherein the liquid composition is present on the wipe in an add-on amount of from about 100% by weight of the treated substrate to about 500% by weight of the treated substrate.

11. The wet wipe of claim 1 wherein the wipe substrate is a nonwoven fibrous sheet material selected from meltblown, coform, air-laid, bonded-carded web materials, hydroentangled materials, and combinations thereof.

12. The wet wipe of claim 1 wherein the emulsifying agent is selected from cetearyl alcohol/dicetyl phosphate/ceteth-10 phosphate blends, PPG-5 ceteth-20, palmitamidopropyltrimonium chloride, polyglyceryl-5 laurate, glyceryl stearate/PEG-100 stearate blends, sorbitan trioleate, sorbitan sesquioleate, hydroxypropyl bis-hydroxyethyldimonium chloride, cetearyl alcohol/PEG-40 castor oil/sodium cetearyl sulfate blends, and combinations thereof.

13. The wet wipe of claim 1 wherein the emulsifying agent has an HLB value between 11 and 20 and acts an oil-in-water emulsifier.

14. The wet wipe of claim 1 wherein the emulsifying agent has an HLB value between 1 and 5 and acts as a secondary emulsifying agent.

15. The wet wipe of claim 1 wherein the acidifying agent is selected from hydrochloric acid, nitric acid, sulfuric acid, phosphoric acid, acetic acid, propanoic acid, citric acid, malic acid, maleic acid, sorbic acid, ascorbic acid, dehydroacetic acid, benzoic acid, chlorobenzoic acid, chloroacetic acid, dichloroacetic acid, trichloroacetic acid, trifluoroacetic acid, lactic acid, glycolic acid, tartaric acid, oxalic acid, acetoacetic acid, betaine, crotonic acid, glyceric acid, dimethylmaleic acid, malonic acid, glutaric acid, succinic acid, dimethylsuccinic acid, adipic acid, azelaic acid, and combinations thereof.

16. The wet wipe of claim 1 further comprising salt.

17. The wet wipe of claim 1 further comprising between 1 and 4% salt.

18. A wet wipe that imparts a perceivable aesthetic benefit to skin, the wipe comprising:
a wipe substrate; and
a liquid composition containing:
from about 75% by weight of the composition to about 99.9% by weight of the composition of water,
from about 0.01% by weight of the composition to about 10% by weight of the composition of potassium soyate;
an acidifying agent selected from hydrochloric acid, nitric acid, sulfuric acid, phosphoric acid, acetic acid, propanoic acid, citric acid, malic acid, maleic acid, sorbic acid, ascorbic acid, dehydroacetic acid, benzoic acid, chlorobenzoic acid, chloroacetic acid, dichloroacetic acid, trichloroacetic acid, trifluoroacetic acid, lactic acid, glycolic acid, tartaric acid, oxalic acid, acetoacetic acid, betaine, crotonic acid, glyceric acid, dimethylmaleic acid, malonic acid, glutaric acid, succinic acid, dimethylsuccinic acid, adipic acid, azelaic acid, and combinations thereof; and
from about 0.01% by weight of the composition to about 10% by weight of the composition of an emulsifying agent selected from cetearyl alcohol/dicetyl phosphate/ceteth-10 phosphate blends, PPG-5 ceteth-20, palmitamidopropyltrimonium chloride, polyglyceryl-5 laurate, glyceryl stearate/PEG-100 stearate blends, sorbitan trioleate, sorbitan sesquioleate, hydroxypropyl bis-hydroxyethyldimonium chloride, cetearyl alcohol/PEG-40 castor oil/sodium cetearyl sulfate blends, and combinations thereof.
